(19) United States
(12) Patent Application Publication
(10) Pub. No.: US 2024/0423727 A1
(43) Pub. Date: Dec. 26, 2024

(54) USING MIXED-REALITY HARDWARE FOR RANGE OF MOTION ESTIMATION DURING ROBOT-ASSISTED ORTHOPEDIC SURGERY

(71) Applicant: Howmedica Osteonics Corp., Mahwah, NJ (US)

(72) Inventor: Jean Chaoui, Lyon (FR)

(21) Appl. No.: 18/684,822

(22) PCT Filed: Aug. 26, 2022
(86) PCT No.: PCT/US2022/041726

§ 371 Date: Feb. 19, 2024

Related U.S. Application Data
(60) Provisional application No. 63/238,767, filed on Aug. 30, 2021.

(51) Int. Cl. A61B 34/30; A61B 34/10; A61B 34/20; A61B 90/00

(52) U.S. Cl. CPC A61B 34/30; A61B 34/10; A61B 34/20; A61B 90/36; A61B 2034/105; A61B 2034/2055; A61B 2034/305; A61B 2090/365

(57) ABSTRACT

A method comprises obtaining position data generated based on signals from one or more sensors of a mixed-reality (MR) visualization device while the bones of the joint are at a plurality of positions; determining, based on the position data, positions of the bones of the joint; generating, by the surgical assistance system, joint tension data based on the positions of the bones of the joint; determining, based on the joint tension data, areas of a target bone to remove, wherein the target bone is one of the bones of tire joint; generating registration data that registers markers with a coordinate system, wherein the markers are attached to one or more of the bones of the joint; and based on die registration data, controlling operation of a robotic arm of a robot during removal of bone tissue from the areas of the target bone.

This application claims priority to U.S. Provisional Patent Application 63/238,767,filed Aug. 30, 2021, the entire content of which is incorporated by reference.

### BACKGROUND

Orthopedic surgeries are complex operations that typically involve a great amount of precision. For example, removing too much or too little bone tissue may have serious implications for whether a patient recovers a full range of motion. Accordingly, robots have been developed to help surgeons perform orthopedic surgeries.

### SUMMARY

This disclosure describes techniques related to robot-assisted orthopedic surgery. For example, one or more markers may be attached to bones of a joint during an orthopedic surgery. A surgical assistance system may generate registration data that registers the markers with a coordinate system. The registration data enables the surgical assistance system to determine a position of a robotic arm of a robot relative to bones of a joint. During the orthopedic surgery, a surgeon may test the movement of the joint in one or more directions. When the surgeon tests the movement of the joint, the patient's anatomy may prevent sensors from detecting the markers, which may cause the surgical assistance system to lose track of the positions of the bones of the joint. Thus, the surgical assistance system may not be able to accurately determine whether to remove additional bone tissue and therefore control the robotic arm accordingly.

This disclosure describes techniques that may address this technical issue. For example, a surgical assistance system may obtain position data, such as depth data, generated based on signals from one or more sensors of a mixed-reality (MR) visualization device while bones of a joint are at a plurality of positions. The surgical assistance system may determine, based on the position data, positions of the bones of the joint. The surgical assistance system may determine joint tension data based on the positions of the bones of the joint. The surgical assistance system may determine, based on the joint tension data, areas of a target bone to remove. Furthermore, the surgical assistance system may generate registration data that registers markers with a coordinate system. Based on the registration data, the surgical assistance system may control operation of a robotic arm of a robot during removal of bone tissue from the areas of the target bone. In this way, by using the position data generated based on signals from one or more sensors of the MR visualization device, the surgical assistance system may be able to track the positions of the bones of the joint while the joint is moved through the plurality of positions. The surgical assistance system may therefore be able to accurately control the robotic arm.

In one example, this disclosure describes a computer-implemented method for assisting an orthopedic surgery, the method comprising: obtaining. by a surgical assistance system, position data generated based on signals from one or more sensors of a mixed-reality (MR) visualization device while bones of a joint are at a plurality of positions, wherein the MR visualization device is worn by a user; determining, by the surgical assistance system, based on the position data, positions of the bones of the joint; generating, by the surgical assistance system, joint tension data based on the positions of the bones of the joint; determining, by the surgical assistance system, based on the joint tension data, areas of a target bone to remove, wherein the target bone is one of the bones of the joint; generating, by the surgical assistance system, registration data that registers markers with a coordinate system, wherein the markers are attached to one or more of the bones of the joint; and based on the registration data, controlling, by the surgical assistance system, operation of a robotic arm of a robot during removal of bone tissue from the areas of the target bone.

In one example, this disclosure describes a surgical assistance system comprising: a memory configured to store registration data; and processing circuitry configured to: obtain position data generated based on signals from one or more sensors of a mixed-reality (MR) visualization device while bones of a joint are at a plurality of positions, wherein the MR visualization device is worn by a user; determine, based on the position data, positions of the bones of the joint; generate joint tension data based on the positions of the bones of the joint; determine, based on the joint tension data, areas of a target bone to remove, wherein the target bone is one of the bones of the joint; generate the registration data, wherein the registration data registers markers with a coordinate system, wherein the markers are attached to one or more of the bones of the joint; and based on the registration data. control operation of a robotic arm of a robot during removal of bone tissue from the areas of the target bone.

The details of various examples of the disclosure are set forth in the accompanying drawings and the description below. Various features, objects, and advantages will be apparent from the description, drawings, and claims.

### BRIEF DESCRIPTION OF DRAWINGS

FIG. 1 is a conceptual diagram illustrating an example operating room arrangement that includes a robot, in accordance with one or more techniques of this disclosure.

FIGS. 2A-2C are conceptual diagrams illustrating ranges of motion of a shoulder joint.

FIG. 3 is a schematic representation of a mixed reality (MR) visualization device in accordance with one or more techniques of this disclosure.

FIG. 4 is a block diagram illustrating an example computing device in accordance with one or more techniques of this disclosure.

FIG. 5 is a conceptual diagram illustrating an example virtual guide overlaid on a target bone of a joint, in accordance with one or more techniques of this disclosure.

FIG. 6 is a user interface showing an example chart of joint tension, in accordance with one or more techniques of this disclosure.

FIG. 7 is a flowchart illustrating an example operation of the surgical assistance system, in accordance with one or more techniques of this disclosure.

### DETAILED DESCRIPTION

During an orthopedic surgery involving a joint of a patient, a surgeon may need to test the range of motion of the joint. To test the range of motion of the joint, the surgeon may move the joint through a range of positions. It may be difficult for sensors mounted on a surgical robot to track the positions of markers attached to bones of the joint while the surgeon is testing the range of motion. In other words, it may be difficult to retain registration between an internal virtual coordinate system and real-world objects, such as the patient's anatomy, while the surgeon is testing the range of motion. Accordingly, it may be difficult for a computing system to generate actionable information based on data generated by the sensors mounted on the surgical robot while the surgeon is testing the range of motion.

This disclosure describes techniques that may address this technical problem. As described in this disclosure, a user, such as a surgeon, may wear a mixed-reality (MR) visualization device that includes its own set of sensors, such as optical sensors and/or depth sensors. A surgical assistance system may obtain position data generated by the sensors of the MR visualization device while the bones of the joint are at various positions. The surgical assistance system may use the position data to determine positions of the bones of the joint at various positions. Based on the positions of the bones, the surgical assistance system may generate joint tension data. The surgical assistance system may use the joint tension data for various purposes, such as determining whether to suggest removing additional bone tissue from one or more of the bones of the joint. After the joint is returned to a position in which the sensors of the surgical robot can reliably detect the positions of bones of the joint, the user may use the surgical robot to perform various actions, such as removing the suggested bone tissue. In this way, by using the sensors of the MR visualization device while the range of motion is being tested. technical problems associated with the surgical robot losing registration may be avoided.

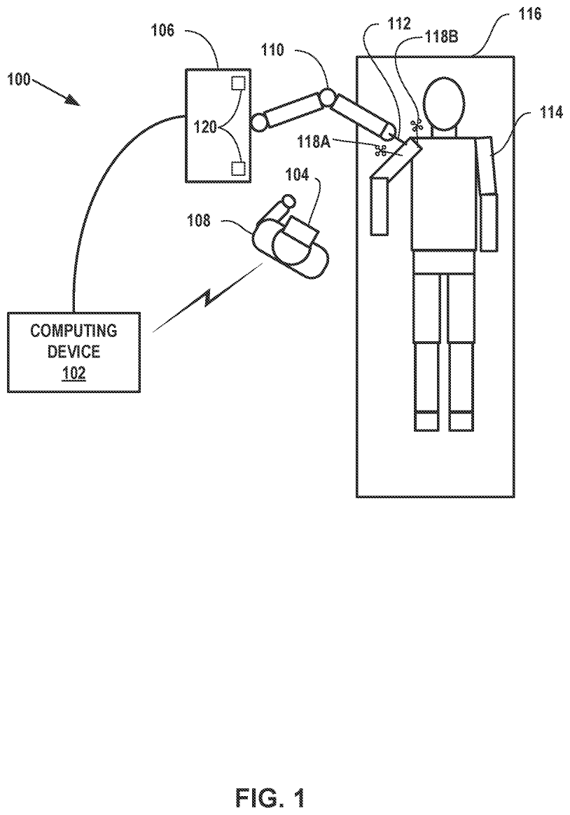

FIG. 1 is a conceptual diagram illustrating an example surgical assistance system 100 that may be used to implement one or more techniques of this disclosure. In the example of FIG. 1, surgical assistance system 100 includes a computing device 102, a MR visualization device 104, and a robot 106. In other examples, surgical assistance system 100 may include more or fewer devices or systems. For instance, in some examples, processing tasks performed by computing device 102 may be performed by MR visualization device 104 and/or robot 106. In other examples, processing tasks described in this disclosure as being performed by computing device 102 may be performed by a system of multiple computing devices. In some examples, processing tasks described in this disclosure as being performed by surgical assistance system 100 may be performed by one or more of computing device 102, MR visualization device 104, robot 106, or one or more other computing devices of surgical assistance system 100. Processing circuitry performing computing tasks of surgical assistance system 100 may be distributed among one or more of computing device 102, MR visualization device 104, robot 106, and/or other computing devices. Furthermore, in some examples, surgical assistance system 100 may include multiple MR visualization devices. Computing device 102 of surgical assistance system 100 may include various types of computing devices, such as server computers, personal computers, smartphones, laptop computers, and other types of computing devices. Computing device 102 may communicate with MR visualization device 104 and robot 106 via one or more wired or wireless communication links.

MR visualization device 104 may use various visualization techniques to display MR visualizations to a user 108, such as a surgeon, nurse, technician, or other type of user. A MR visualization may comprise one or more virtual objects that are viewable by a user at the same time as real-world objects. Thus, what the user sees is a mixture of real and virtual objects. User 108 does not form part of surgical assistance system 100.

MR visualization device 104 may comprise various types of devices for presenting MR visualizations. For instance, in some examples, MR visualization device 104 may be a Microsoft HOLOLENS™ headset, such as the HOLOLENS 2 headset. available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment, through the holographic lenses. In some examples, MR visualization device 104 may be a holographic projector, head-mounted smartphone, special-purpose MR visualization device, or other type of device for presenting MR visualizations. In some examples, MR visualization device 104 includes a head-mounted unit and a backpack unit that performs at least some of the processing functionality of MR visualization device 104. In other examples, all functionality of MR visualization device 104 is performed by hardware residing in a head-mounted unit. Discussion in this disclosure of actions performed by surgical assistance system 100 may be performed by one or more computing devices (e.g., computing device 102) of surgical assistance system 100, MR visualization device 104. or a combination of the one or more computing devices and MR visualization device 104.

Robot 106 includes a robotic arm 110. In some examples, robot 106 may be a MAKO robot from Stryker Corporation of Kalamazoo, Michigan. A surgical tool 112 is connected to robotic arm 110. Surgical tool 112 may comprise a cutting burr, scalpel, drill. saw, or other type of tool that may be used during surgery. Robot 106 may control robotic arm 110 to change the position of surgical tool 112.

In the example of FIG. 1, a patient 114 lies on a surgical table 116 and is undergoing an orthopedic shoulder surgery. In other examples, the techniques of this disclosure may be applied with respect to orthopedic surgeries on other parts of the body of patient 114, such as the knee, hip, spine, elbow, ankle, foot, hand, and so on. Patient 114 does not form part of surgical assistance system 100.

In the example of FIG. 1, markers 118A, 118B (collectively, “markers 118”) are attached to bones of patient 114. For instance, in the example of FIG. 1, marker 118A may be attached to a humerus of patient 114 and marker 118B may be attached to a scapula of patient 114. Each of markers 118 may comprise a plurality of facets. For instance, in one example, one or more of markers 118 may include a cube that bas six evenly sized facets. In another example, one or more of markers 118 may have another shape, such as a pyramidal shape or a polyhedral shape. In some examples, markings may be formed on the facets of markers 118. Such markers may include one or more of QR codes, bar codes, images, text, or other markings. There may be different markings on different facets of markers 118. The markings on different facets of markers 118 may serve to visually identify the different facets of markers 118. In some examples, markers 118 may be or include electromagnetic markers.

One or more sensors 120 may be included in robot 106 or elsewhere in the environment of robot 106. Sensors 120 may include video cameras, depth sensors, or other types of sensors. Computing device 102 is configured to use signals (e.g., images, point clouds, etc.) from sensors 120 to perform a registration operation that registers positions of virtual objects with the positions of markers 118. The virtual objects may include models of one or more bones of patient 114 shaped in accordance with a surgical plan. By registering the virtual objects with the positions of markers 118, computing device 102 may be able to relate the virtual objects with the positions of markers 118. Because the positions of the bones of patient 114 are connected to markers 118, registering the virtual objects with the positions of markers 118 may enable computing device 102 to determine positions of the virtual objects relative to the positions of the bones of patient 114. Thus, computing device 102 may be able to determine whether surgical tool 112 is being used in accordance with the surgical plan.

During the orthopedic surgery, a surgeon (e.g., user 108), may attach one or more trial prostheses to bones of a joint of patient 114. For instance, in the example of FIG. 1. the surgeon may attach trial prostheses to a humerus and a scapula of patient 114. In examples where the orthopedic surgery is an anatomic shoulder replacement surgery, a trial prosthesis attached to the humerus of patient 114 includes a ball-shaped member that moves within a cup-shaped member of a trial prosthesis attached to a scapula of patient 114. In examples where the orthopedic surgery is a reverse shoulder replacement surgery, a trial prosthesis attached to the scapula of patient 114 includes a ball-shaped member that moves within a cup-shaped member of a trial prosthesis attached to a humerus of patient 114. In other examples, the surgeon may attach trial prostheses to a femur and tibia of patient 114.

In some examples, user 108 may use robot 106 to perform one or more parts of a process to install a trial prosthesis. For instance, in an example where surgical tool 112 is a cutting burr, user 108 may guide surgical tool 112 to a bone of patient 114 and use surgical tool 112 to remove areas of the bone necessary for installation of the trial prosthesis. In this example, during the process of removing the areas of the bone, robot 106 may respond to efforts by user 108 to remove areas of the bone determined in a surgical plan to remain with the bone. In an example where the surgical tool 112 is a drill, user 108 may use surgical tool 112 to drill a hole in a bone of patient 114. In this example, robot 106 may respond to efforts by user 108 to drill the hole at an angle or position that is not in accordance with a surgical plan. In these examples, computing device 102 uses the registration data to determine the position of robot 106 (and surgical tool 112) in order to determine whether to respond to movement of surgical tool 112 by user 108. Responding to a movement of surgical tool 112 may involve robot 106 providing haptic feedback, robot 106 providing counteracting force via robotic arm 110 to the movement of surgical tool 112, generating audible or visible indications, and/or performing other actions. In some examples, robot 106 may guide surgical tool 112 while user 108 supervises operation of robot 106. In such examples, the hand of user 108 may rest on surgical tool 112 as robot 106 moves surgical tool 112 and user 108 may stop the movement of surgical tool 112 if user 108 is concerned that robot 106 has moved surgical tool 112 to an inappropriate location. In some examples, user 108 does not touch surgical tool 112 while robot 106 is maneuvering surgical tool 112. Rather, in some such examples, user 108 may use a physical or virtual controller to stop robot 106 from moving surgical tool 112 if user 108 determines that robot 106 has moved surgical tool 112 to an inappropriate location.

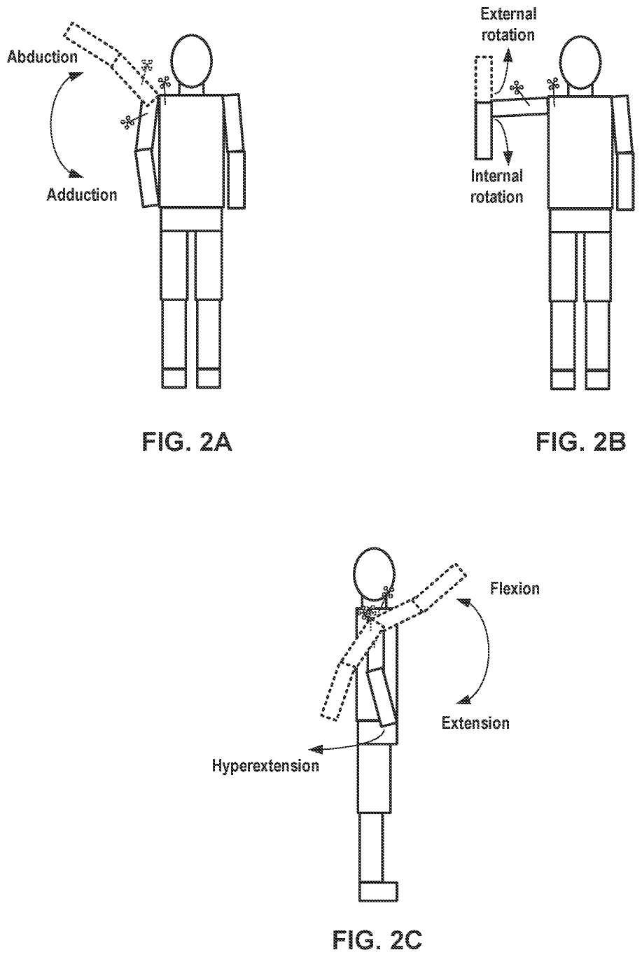

After attaching the trial prostheses to the bones of the joint of patient 114, user 108 may test whether patient 114 has an appropriate range of motion. To test whether patient 114 has an appropriate range of motion, user 108 may move the joint in one or more directions. In some instances, to move the joint, user 108 may move a body part of patient 114 associated with the joint. For instance, in the example of FIG. 1 user 108 may move the right shoulder joint of patient 114 by moving the right arm of patient 114. FIGS. 2A-2C are conceptual diagrams illustrating ranges of motion of a shoulder joint. Specifically, FIG. 2A illustrates abduction and adduction, FIG. 2B illustrates external rotation and internal rotation, and FIG. 2C illustrates flexion, extension, and hyperextension. In other examples, such as examples where the orthopedic surgery is being performed on a knee of patient 114, user 108 may move a leg of patient 114 containing the knee.

Patient 114 may not have an appropriate range of motion if the tension on the joint is not proper. For instance, if the tension in the joint is too loose, the joint may allow excessive motion in one or more directions, which may lead to instability of the joint. If the tension in the joint is too tight. the joint may not be able to achieve a normal range of motion. which may limit the mobility of patient 114. Typically, the tension on the joint is too loose if there is too much space between the bones of the joint. Similarly, the tension on the joint is typically too tight if there is not enough space between the bones of the joint. Looseness may be addressed by using a larger prosthesis that reduces the amount of space between the bones of the joint and/or adjusting a position of the prosthesis. Tightness may be addressed by using a smaller prosthesis, adjusting a position of the prosthesis, and/or removing additional bone.

During the process of testing whether patient 114 has the appropriate range of motion, portions of the anatomy of patient 114 may obscure markers 118 from the perspective of sensors 120 used by computing device 102 to determine the position of robot 106 relative to patient 114. In other words, portions of the anatomy of patient 114 may come between markers 118 and sensors 120. Moreover, even if markers 118 are not obscured, computing device 102 may be unable to accurately determine the range of motion of patient 114 based on the positions of markers 118. Accurately determining positions of the bones of the joint while user 108 is testing the range of motion of the joint may be important in examples where computing device 102 determines the tension of the joint by simulating the motion of the bones using 3D models of the bones and determining distances between the 3D models of the bones.

This disclosure describes techniques that may address one or more of these technical problems. In this disclosure. surgical assistance system 100 may obtain position data generated based on signals from one or more sensors of a MR visualization device 104 while the bones of the joint are at a first plurality of positions. As shown in the example of FIG. 1, MR visualization device 104 may be worn by user 108. Furthermore, surgical assistance system 100 may determine, based on the position data, positions of the bones of the joint Surgical assistance system 100 may generate joint tension data based on the positions of the bones of the joint. Additionally, surgical assistance system 100 may determine, based on the joint tension data, areas of a target bone to remove. The target bone is one of the bones of the joint. Additionally, surgical assistance system 100 may generate registration data that registers markers 118 with a coordinate system. Markers 118 are attached to one or more of the bones of the joint. Based on the registration data, surgical assistance system 100 may control operation of robotic arm 110 during removal of bone tissue from the areas of the target bone.

The techniques of this disclosure may be applicable to various bones. For example, the techniques of this disclosure may be applicable to a scapula of the patient, a humerus of the patient, a fibula of the patient, a patella of the patient, a tibia of the patient, a talus of the patient, a hip of the patient, a femur of the patient, or another type of bone of the patient.

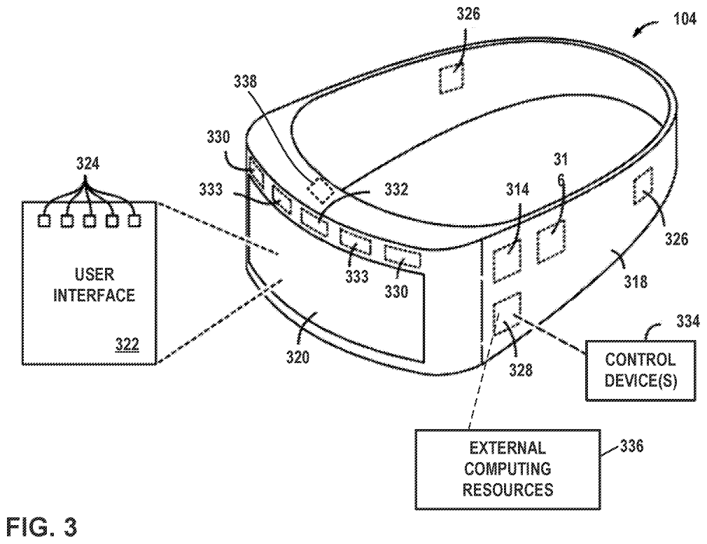

FIG. 3 is a schematic representation of MR visualization device 104 in accordance with one or more techniques of this disclosure. As shown in the example of FIG. 3, MR visualization device 104 can include a variety of electronic components found in a computing system, including one or more processor(s) 314 (e.g., microprocessors or other types of processing units) and memory 316 that may be mounted on or within a frame 318. Furthermore, in the example of FIG. 3, MR visualization device 104 may include a transparent screen 320 that is positioned at eye level when MR visualization device 104 is worn by a user. In some examples, screen 320 can include one or more liquid crystal displays (LCDs) or other types of display screens on which images are perceptible to a user who is wearing or otherwise using MR visualization device 104 via screen 320. Other display examples include organic light emitting diode (OLED) displays. In some examples, MR visualization device 104 can operate to project 3D images onto the user's retinas using techniques known in the art.

In some examples, screen 320 may include see-through holographic lenses. sometimes referred to as waveguides, that permit a user to see real-world objects through (e.g., beyond) the lenses and also see holographic imagery projected into the lenses and onto the user's retinas by displays, such as liquid crystal on silicon (LCoS) display devices, which are sometimes referred to as light engines or projectors. operating as an example of a holographic projection system 338 within MR visualization device 104. In other words, MR visualization device 104 may include one or more see-through holographic lenses to present virtual images to a user. Hence, in some examples, MR visualization device 104 can operate to project 3D images onto the user's retinas via screen 320, e.g., formed by holographic lenses. In this manner, MR visualization device 104 may be configured to present a 3D virtual image to a user within a real-world view observed through screen 320, e.g., such that the virtual image appears to form part of the real-world environment. In some examples, MR. visualization device 104 may be a Microsoft HOLOLENS™ headset, available from Microsoft Corporation, of Redmond, Washington, USA, or a similar device, such as, for example, a similar MR visualization device that includes waveguides. The HOLOLENS™ device can be used to present 3D virtual objects via holographic lenses, or waveguides, while permitting a user to view actual objects in a real-world scene, i.e., in a real-world environment. through the holographic lenses.

Although the example of FIG. 3 illustrates MR visualization device 104 as a head-wearable device, MR visualization device 104 may have other forms and form factors. For instance, in some examples, MR visualization device 104 may be a handheld smartphone or tablet.

MR visualization device 104 can also generate a user interface (UI) 322 that is visible to the user, e.g., as holographic imagery projected into see-through holographic lenses as described above. For example, UI 322 can include a variety of selectable widgets 324 that allow the user to interact with a MR system. Imagery presented by MR visualization device 104 may include. for example, one or more 2D or 3D virtual objects. MR visualization device 104 also can include a speaker or other sensory devices 326 that may be positioned adjacent the user's ears. Sensory devices 326 can convey audible information or other perceptible information (e.g., vibrations) to assist the user of MR visualization device 104.

MR visualization device 104 can also include a transceiver 328 to connect MR visualization device 104 to a network, a computing cloud, such as via a wired communication protocol or a wireless protocol, e.g., Wi-Fi, Bluetooth, etc. MR visualization device 104 also includes a variety of sensors to collect sensor data. such as one or more optical sensor(s) 330 and one or more depth sensor(s) 332 (or other depth sensors), mounted to, on or within frame 318. In some examples, optical sensor(s) 330 are operable to scan the geometry of the physical environment in which user 108 is located (e.g., an operating room) and collect two-dimensional (2D) optical image data (either monochrome or color). Depth sensor(s) 332 are operable to provide 3D image data, such as by employing time of flight, stereo or other known or future-developed techniques for determining depth and thereby generating image data in three dimensions. Other sensors can include motion sensors 333 (e.g., Inertial Mass Unit (IMU) sensors, accelerometers, etc.) to assist with tracking movement.

Surgical assistance system 100 (e.g., computing device 102) may process the sensor data so that geometric, environmental, textural, or other types of landmarks (e.g., corners. edges or other lines. walls. floors. objects) in the user's environment or “scene” can be defined and movements within the scene can be detected. As an example, the various types of sensor data can be combined or fused so that the user of MR visualization device 104 can perceive virtual objects that can be positioned, or fixed and/or moved within the scene When a virtual object is fixed in the scene, the user can walk around the virtual object, view the virtual object from different perspectives, and manipulate the virtual object within the scene using hand gestures, voice commands, gaze line (or direction) and/or other control inputs. In some examples, the sensor data can be processed so that the user can position a 3D virtual object (e.g., a bone model) on an observed physical object in the scene (e.g., a surface, the patient's real bone, etc.) and/or orient the 3D virtual object with other virtual objects displayed in the scene. In some examples, surgical assistance system 100 may process the sensor data so that user 108 can position and fix a virtual representation of the surgical plan (or other widget, image or information) onto a surface, such as a wall of the operating room. Yet further, in some examples, surgical assistance system 100 may use the sensor data to recognize surgical instruments and determine the positions of those instruments.

MR visualization device 104 may include one or more processors 314 and memory 316, e.g., within frame 318 of MR visualization device 104. In some examples, one or more external computing resources 336 process and store information, such as sensor data, instead of or in addition to in-frame processor(s) 314 and memory 316. For example, external computing resources 336 may include processing circuitry, memory, and/or other computing resources of computing device 102 (FIG. 1). In this way, data processing and storage may be performed by one or more processors 314 and memory 316 within MR visualization device 104 and/or some of the processing and storage requirements may be offloaded from MR visualization device 104. Hence, in some examples, one or more processors that control the operation of MR visualization device 104 may be within MR visualization device 104, e.g., as processor(s) 314. Alternatively, in some examples, at least one of the processors that controls the operation of MR visualization device 104 may be external to MR visualization device 104, e.g., as processor(s) 314. Likewise, operation of MR visualization device 104 may, in some examples, be controlled in part by a combination of one or more processors 314 within the visualization device and one or more processors external to MR visualization device 104.

For instance, in some examples, when MR visualization device 104 is in the context of FIG. 3, processing of the sensor data can be performed by processor(s) 314 in conjunction with memory or storage device(s) 315. In some examples, processor(s) 314 and memory 316 mounted to frame 318 may provide sufficient computing resources to process the sensor data collected by optical sensor(s) 330, depth sensor(s) 332 and motion sensors 333. In some examples, the sensor data can be processed using a Simultaneous Localization and Mapping (SLAM) algorithm, or other algorithms for processing and mapping 2D and 3D image data and tracking the position of MR system, e.g., by one or more sensors and processors 314 within a MR visualization device 104 substantially conforming to the Microsoft HOLOLENS™ device or a similar mixed reality (MR) visualization device.

In some examples, surgical assistance system 100 can also include user-operated control device(s) 334 that allow user 108 to operate MR visualization device 104, use MR visualization device 104 in spectator mode (either as master or observer), interact with UI 322 and/or otherwise provide commands or requests to processors(s) 314 or other systems connected to a network. As examples, control device(s) 334 can include a microphone, a touch pad, a control panel, a motion sensor or other types of control input devices with which the user can interact.

As described in this disclosure, surgical assistance system 100 may use data from sensors of MR visualization device 104 (e.g., optical sensor(s) 330, depth sensor(s) 332, etc.) to track the positions of markers 118 while user 108 tests the range of motion of the joint of patient 114. Because the sensors of MR visualization device 104 are mounted on MR visualization device 104, user 108 may move the sensors of MR visualization device 104 to positions in which markers 118 are not obscured from the view of the sensors of MR visualization device 104. Surgical assistance system 100 may determine the positions of the bones based on the positions of markers 118. Surgical assistance system 100 may then generate joint tension data based on the positions of the bones. In some examples, surgical assistance system 100 may determine areas of a target bone to remove based on the joint tension data.

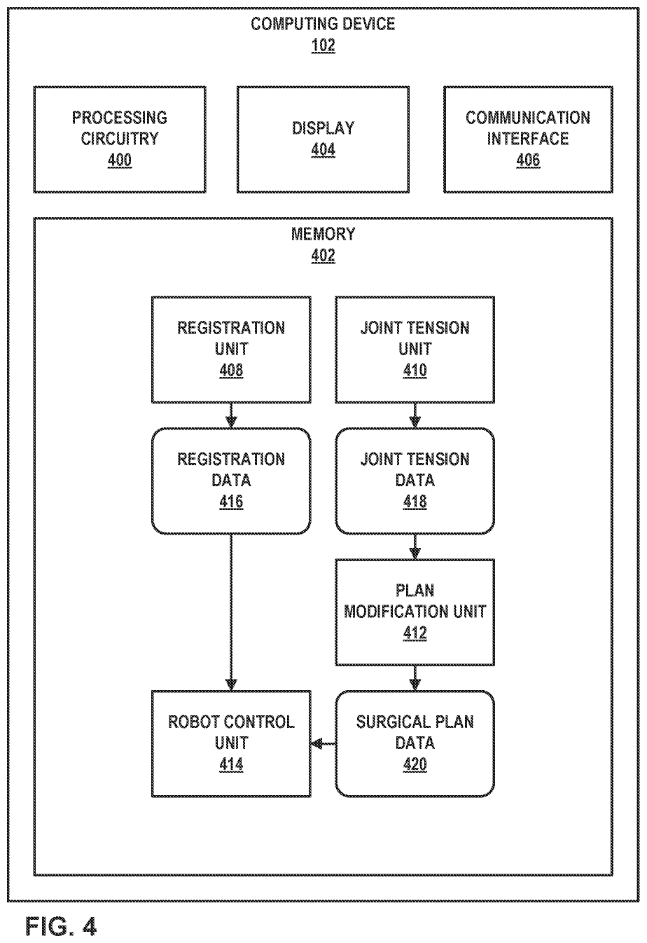

FIG. 4 is a block diagram illustrating an example computing device in accordance with one or more techniques of this disclosure. In the example of FIG. 4, computing device 102 includes processing circuitry 400, memory 402, display 404, and a communication interface 406. Display 404 is optional, such as in examples where computing device 102 comprises a server computer.

Examples of processing circuitry 400 include one or more microprocessors. digital signal processors (DSPs), application specific integrated circuits (ASICs), field programmable gate arrays (FPGAs), hardware, or any combinations thereof. In general, processing circuitry 400 may be implemented as fixed-function circuits, programmable circuits, or a combination thereof. Fixed-function circuits refer to circuits that provide particular functionality and are preset on the operations that can be performed. Programmable circuits refer to circuits that can be programmed to perform various tasks and provide flexible functionality in the operations that can be performed. For instance, programmable circuits may execute software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. In some examples, the one or more of the units may be distinct circuit blocks (fixed-function or programmable), and in some examples, the one or more units may be integrated circuits.

Processing circuitry 400 may include arithmetic logic units (ALUs), elementary function units (EFUs), digital circuits, analog circuits, and/or programmable cores, formed from programmable circuits In examples where the operations of processing circuitry 400 are performed using software executed by the programmable circuits, memory 402 may store the object code of the software that processing circuitry 400 receives and executes, or another memory within processing circuitry 400 (not shown) may store such instructions. Examples of the software include software designed for surgical planning. Processing circuitry 400 may perform the actions ascribed in this disclosure to surgical assistance system 100.

Communication interface 406 of computing device 102 allows computing device 102 to output data and instructions to and receive data and instructions from MR visualization device 104 and/or robot 106. Communication interface 406 may be hardware circuitry that enables computing device 102 to communicate (e.g., wirelessly or using wires) with other computing systems and devices, such as MR visualization device 104. The network may include various types of communication networks including one or more wide-area networks. such as the Internet, local area networks, and so on. In some examples, the network may include wired and/or wireless communication links.

Memory 402 may be formed by any of a variety of memory devices, such as dynamic random access memory (DRAM), including synchronous DRAM (SDRAM), magnetoresistive RAM (MRAM), resistive RAM (RRAM), or other types of memory devices. Examples of display 404 may include a liquid crystal display (LCD), a plasma display, an organic light emitting diode (OLED) display, or another type of display device.

Memory 402 may store various types of data used by processing circuitry 400. For example, memory 402 may store data describing 3D models of various anatomical structures, including morbid and predicted premorbid anatomical structures. For instance, in one specific example, memory 402 may store data describing a 3D model of a humerus of a patient, imaging data, and other types of data.

In the example of FIG. 4, memory 402 contains a registration unit 408. a joint tension unit 410, a plan modification unit 412, and a robot control unit 414. Registration unit 408, joint tension unit 410, a plan modification unit 412, and robot control unit 414 may comprise software that is executable by processing circuitry 400. In other words, the software of registration unit 408, joint tension unit 410, a plan modification unit 412, and robot control unit 414 may configure processing circuitry 400 to perform the actions ascribed in this disclosure to registration unit 408, joint tension unit 410, plan modification unit 412, and robot control unit 414. Furthermore, in the example of FIG. 4, memory 402 may include registration data 416, joint tension data 418. and surgical plan data 420. Although FIG. 4 shows registration unit 408, joint tension unit 410, plan modification unit 412, robot control unit 414, registration data 416, joint tension data 418, and surgical plan data 420 in memory 402 of computing device 102, one or more of registration unit 408, joint tension unit 410, plan modification unit 412, robot control unit 414. registration data 416, joint tension data 418, and surgical plan data 420 may be fully or partly included in one or more other devices of surgical assistance system 100, such as robot 106 or MR visualization device 104.

Registration unit 408 may perform a registration process that uses data from one or more of sensors 120 to determine a spatial relationship between virtual objects and real-world objects. In other words, by performing the registration process, registration unit 408 may generate registration data that describes a spatial relationship between one or more virtual objects and real-world objects. The virtual objects include a model of a bone that is shaped in accordance with the surgical plan. The registration data may express a transformation function that maps a coordinate system of the virtual objects to a coordinate system of the real-world objects. In some examples, the registration data is expressed in the form of a transform matrix that, when multiplied by a coordinate of a point in the coordinate system of the real-world objects, results in a coordinate of a point in the coordinate system of the virtual objects.

As part of performing the registration process, registration unit 408 may generate a first point cloud and a second point cloud. The first point cloud includes points corresponding to landmarks on one or more virtual objects, such as a model of a bone. The second point cloud includes points corresponding to landmarks on real-world objects, such as markers 118. Landmarks may be locations on virtual or real-world objects. The points in the first point cloud may be expressed in terms of coordinates in a first coordinate system and the points in the second point cloud may be expressed in terms of coordinates in a second coordinate system. Because the virtual objects may be designed with positions that are relative to one another but not relative to any real-world objects, the first and second coordinate systems may be different.

Registration unit 408 may generate the second point cloud using a Simultaneous Localization and Mapping (SLAM) algorithm. By performing the SLAM algorithm, registration unit 408 may generate the second point cloud based on observation data generated by sensors 120. Registration unit 408 may perform one of various implementations of SLAM algorithms, such as a SLAM algorithm having a particular filter implementation, an extended Kalman filter implementation, a covariance intersection implementation, a GraphSLAM implementation, an ORB SLAM implementation, or another implementation. In accordance with some examples of this disclosure, registration unit 408 may apply an outlier removal process to remove outlying points in the first and/or second point clouds. In some examples, the outlying points may be points lying beyond a certain standard deviation threshold from other points in the point clouds. Applying outlier removal may improve the accuracy of the registration process.

In some examples, as part of performing the registration process, registration unit 408 may apply an image recognition process that uses information from sensors 120 to identify markers 118. Identifying markers 118 may enable registration unit 408 to determine a preliminary spatial relationship between points in the first point cloud and points in the second point cloud. The preliminary spatial relationship may be expressed in terms of translational and rotational parameters.

Next, registration unit 408 may refine the preliminary spatial relationship between points in the first point cloud and points in the second point cloud. For example, registration unit 408 may perform an iterative closest point (ICP) algorithm to refine the preliminary spatial relationship between the points in the first point cloud and the points in the second point cloud. The iterative closest point algorithm finds a combination of translational and rotational parameters that minimize the sum of distances between corresponding points in the first and second point clouds. For example, consider a basic example where landmarks corresponding to points in the first point cloud are at coordinates A, B, and C and the same landmarks correspond to points in the second point cloud are at coordinates A′, B′, and C″. In this example, the iterative closest point algorithm determines a combination of translational and rotational parameters that minimizes Δ+ΔB+ΔC, where ΔA is the distance between A and A′, ΔB is the distance between B and B′, and ΔC is the distance between C and C′. To minimize the sum of distances between corresponding landmarks in the first and second point clouds, registration unit 408 may perform the following steps:
1. For each point of the first point cloud, determine a corresponding point in the second point cloud. The corresponding point may be a closest point in the second point cloud. In this example, the first point cloud includes points corresponding to landmarks on one or more virtual objects and the second point cloud may include points corresponding to landmarks on real-world objects (e.g., markers 118).
2. Estimate a combination of rotation and translation parameters using a root mean square point-to-point distance metric minimization technique that best aligns each point of the first point cloud to its corresponding point in the second point cloud.
3. Transform the points of the first point cloud using the estimated combination of rotation and translation parameters.
4. Iterate steps 1-3 using the transformed points of the first point cloud.

In this example, after performing an appropriate number of iterations, registration unit 408 may determine rotation and translation parameters that describe a spatial relationship between the original positions of the points in the first point cloud and the final positions of the points in the first point cloud. The determined rotation and translation parameters can therefore express a mapping between the first point cloud and the second point cloud. Registration data 416 may include the determined rotation and translation parameters. In this way, registration unit 408 may generate registration data 416.

As mentioned above, user 108 may test the range of motion of a joint of patient 114 during an orthopedic surgery. For instance. user 108 may test the range of motion of the joint after attaching one or more trial prostheses to the bones of the joint. While user 108 is testing the range of motion of the joint, sensors of MR visualization device 104 (e.g,, optical sensor(s) 330, depth sensor(s) 332, etc.) may track the motion of markers 118. Joint tension unit 410 may use data from the sensors of MR visualization device 104 to determine positions of bones of the joint. For instance, in the example of FIG. 1, joint tension unit 410 may determine positions of a humerus and scapula of patient 114. In some examples. to determine the positions of the bones of the joint, intra-operative images, such as x-ray images, may be captured of the joint after markers 118 are attached to the bones of the joint. Because markers 118 maintain a fixed position relative to bones during the orthopedic surgery and because joint tension unit 410 may use the medical images to determine the relative positions of the bones and markers 118, joint tension unit 410 may use data from sensors of MR visualization device 104 to determine positions of the bones based on the positions of markers 118 while user 108 tests the range of motion of the joint.

Joint tension unit 410 may generate joint tension data 418 based on the positions of the bones while user 108 tests the range of motion of the joint. Joint tension data 418 may include data indicating distances between the bones at various points in the range of motion. For example, joint tension data 418 may include data indicating that the minimum distance between the scapula and humerus (or scapula and/or humeral prostheses) may be 1 millimeter when the arm of patient 114 is abducted to 45° and may be 0.1 millimeter when abducted to 70°. Joint tension unit 410 may determine the distances in joint tension data 418 during virtual movements of models of the bones (and potentially prostheses attached thereto) that emulate the positions of the bones determined based on the sensors of MR visualization device 104. The distance between the bones is indicative of the tension of the joint because greater distances between the bones relate to looser tension in the joint and smaller distances between the bones relates to greater tension in the joint.

Surgical plan data 420 may include data indicating a planned shape of the bones of the joint and prostheses attached to the bones. For instance, surgical plan data 420 may indicate data indicating bone shapes and prostheses present when user 108 tests the range of motion of the joint. Plan modification unit 412 may identify one or more changes to make to surgical plan data 420 based on joint tension data 418. For example, plan modification unit 412 may identify a differently sized prosthesis to use in the joint and/or different position of the prosthesis. For instance, in a shoulder surgery, plan modification unit 412 may modify surgical plan data 420 to use an inferior offset of a glenoid prosthesis if the distance between the humerus (or humeral prosthesis) and scapula (or glenoid prosthesis) is too small at the top of the range of motion during abduction of the arm of patient 114. In some examples where the distance is too great at one or more points, plan modification unit 412 may identify a larger prosthesis to use.

In some examples, plan modification unit 412 may determine, based on joint tension data 418, that additional bone tissue should be removed from a bone (e.g., a target bone) of the joint. For instance, based on the distance between the bones being too small at one or more points, plan modification unit 412 may determine that additional bone tissue should be removed so that the position of a prosthesis can be modified to allow for more space between the bones. In examples where plan modification unit 412 suggests removal of additional bone tissue, surgical assistance system 100 (e.g., MR visualization device 104 or display 404 of computing device 102) may output an indication regarding the suggestion to remove the additional bone tissue. Plan modification unit 412 may update surgical plan data 420 in response to receiving an indication of user input to accept the suggestion. For instance, plan modification unit 412 may update a 3D virtual model of the target bone such that the 3D virtual model excludes the bone tissue planned for removal.

In examples where plan modification unit 412 updates surgical plan data 420 to indicate removal of additional bone tissue from the target bone, user 108 may use surgical tool 112 to remove the additional bone tissue from the target bone. In some examples. robotic arm 110 may respond to attempts, accidentally or otherwise, by user 108 to shape the target bone into a form inconsistent with the 3D virtual model of the target bone. Thus, robotic arm 110 may enable user 108 to precisely shape the target bone with lower likelihood of error. In another example, user 108 may use surgical tool 112 to insert a drill bit, screw, pin, or other surgical item into the target bone. In this example, robotic arm 110 may respond to attempts by user 108 to insert the surgical item into the target bone at an angle inconsistent with a planned angle. Robot 106 may use registration data 416 to determine the spatial relationship between surgical tool 112 and the target bone. User 108 may override or ignore responses of robotic arm 110 if user 108 determines that doing so is desirable. For example, user 108 may override the response of robotic arm 110 by increasing pressure on surgical tool 112, providing input to surgical assistance system 100 (e.g., via a hand gesture, voice command, etc.) to override the response, and/or by performing other actions. While user 108 is using surgical tool 112, MR visualization device 104 may present to user 108 one or more virtual guides to help user 108 use surgical tool 112. For instance. MR visualization device 104 may present to user 108 a virtual guide indicating areas of the target bone to remove, an angle of insertion of a surgical item into the target bone, and so on.

In some examples, robotic arm 110 may move surgical tool 112 under the supervision of user 108 but without user 108 guiding movement of surgical tool 112. In such examples, the hand of user 108 may rest on surgical tool 112 as robotic arm 110 moves surgical tool 112 and user 108 may intervene if robotic arm 110 moves surgical tool 112 to a position not desired by user 108. In other examples, user 108 does not touch surgical tool 112 or robotic arm 110 while robotic arm 110 moves surgical tool 112. In some examples where robotic arm 110 moves surgical tool 112 under the supervision of user 108, MR visualization device 104 may present to user 108 a virtual guide associated with the actions being performed by robot 106. For instance, the virtual guide may show areas of the target bone to be removed. In another example, the virtual guide may indicate a planned angle of insertion of a drill bit, pin, screw, or other surgical item. In this example, user 108 may use the virtual guide to check whether robotic arm 110 is inserting the surgical item into the target bone at the planned angle of insertion. Examples of virtual guides may include one or more virtual axes, virtual planes, virtual targets, textual indications, other indications of trajectory presented by MR visualization device 104, which may be in combination with audible or haptic feedback in some examples.

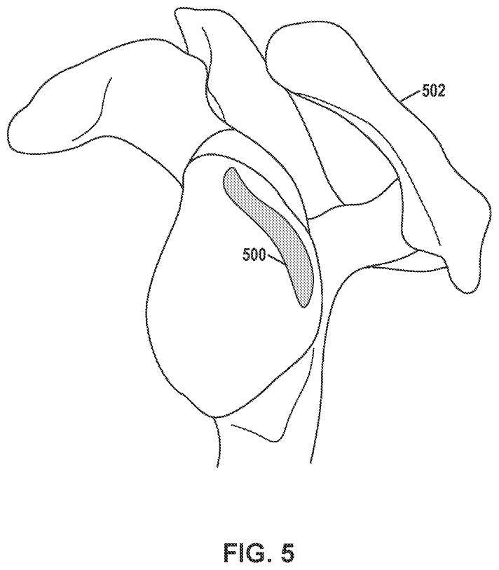

FIG. 5 is a conceptual diagram illustrating an example virtual guide 500 overlaid on a target bone 502 of a joint, in accordance with one or more techniques of this disclosure. In the example of FIG. 5, target bone 502 is a scapula shown from a lateral perspective. Other equivalent examples may apply with respect to other types of bones. FIG. 5 shows more parts of the scapula than would be exposed during an actual shoulder surgery. Based on joint tension data 418, plan modification unit 412 may suggest removal of areas (e.g., 2D or 3D zones) of bone tissue from target bone 502. To assist user 108 in removing the areas of bone tissue from target bone 502, MR visualization device 104 may present virtual guide 500 to indicate to user 108 the bone tissue to remove from target bone 502. Virtual guide 500 may be a virtual object, such as a semi-transparent shaded or colored area, an outline, or other form that indicates the bone tissue to remove from target bone 502. In some examples, areas within virtual guide 500 may be color coded to indicate how much bone tissue to remove within the areas. MR visualization device 104 may present virtual guide 500 to user 108 while user 108 is using surgical tool 112 (which is connected to robotic arm 110) to remove the bone tissue from target bone 502. In some examples, surgical assistance system 100 may cause an MR visualization device other than MR visualization device 104 (e.g., a second MR visualization device) to present virtual guide 500.

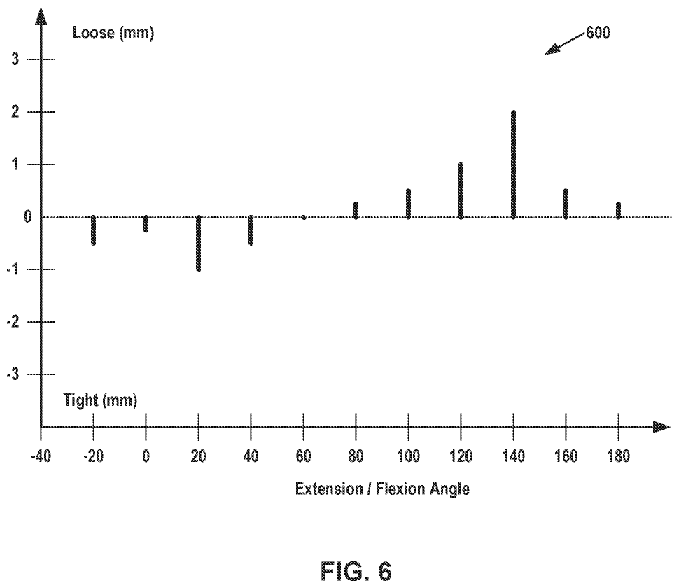

FIG. 6 is a user interface 600 showing an example chart of joint tension data 418, in accordance with one or more techniques of this disclosure. Computing device 102, MR visualization device 104, or another device of surgical assistance system 100 may display user interface 600 to user 108 or another person during an orthopedic surgery. User interface 600 may help user 108 understand the tension of a joint (e.g., a shoulder joint in the example of FIG. 6) during a movement of the joint (e.g., extension and flexion in the example of FIG. 6). Joint tension unit 410 may generate similar charts for other directions of movement.

The vertical bars shown in user interface 600 show, for various angles, a difference between a minimum distance between the bones and a targeted distance between the bones. In this disclosure, discussion of distance between bones may apply to distances between two bones, distances between a bone and a prosthesis attached to another bone, or distances between prostheses attached to two separate bones. Thus, in the example of FIG. 6, the joint is loose by 2 millimeters (mm) at 140° and tight by 1 mm at 20°.

Plan modification unit 412 may use the joint tension data 418 represented in the chart of FIG. 6 to generate suggestions for modifying surgical plan data 420. For instance, plan modification unit 412 may use mapping data that maps joint tension data 418, such as the type of data shown in FIG. 6, to suggestions for modifying surgical plan data 420. In some examples, plan modification unit 412 includes a machine-learned model (e.g., an artificial neural network, a support vector machine (SVM), a k-means clustering algorithm, etc.) to determine a suggestion based on joint tension data 418, such as the type of data shown in FIG. 6.

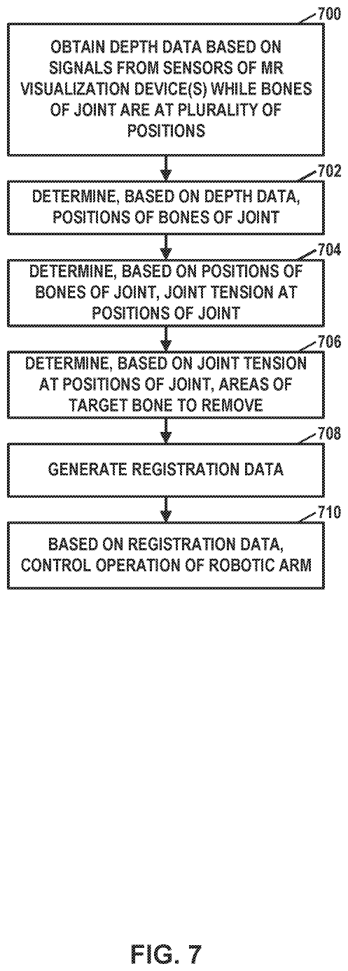

FIG. 7 is a flowchart illustrating an example operation of surgical assistance system 100, in accordance with one or more techniques of this disclosure. Other operations of surgical assistance system 100 in accordance with the techniques of this disclosure may involve more, fewer, or different actions.

In the example of FIG. 7, surgical assistance system 100 (e.g., joint tension unit 410) may obtain position data generated based on signals from one or more sensors (e.g., optical sensor(s) 330, depth sensor(s) 332. etc.) of MR visualization device 104 while the bones of the joint are at a plurality of positions (700). The plurality of positions may be positions along a direction of motion, such as abduction/adduction, external rotation/internal rotation, flexion/extension, etc. MR visualization device 104 may be worn by user 108. The position data may indicate positions of physical markers 118, which may be tracked by sensors 120 and may have a fixed spatial relationship with the bones of the joint. In some examples, MR visualization device 104 performs a registration process that relates virtual models of the bones of the joint to the actual bones of the joint. The registration process may be the same or similar to the registration process described above with respect to registration unit 408.

Furthermore, surgical assistance system 100 (e.g., joint tension unit 410) may determine, based on the position data, positions of the bones of the joint (702). For example, surgical assistance system 100 may store 3D virtual models of the bones of the joint. The 3D virtual models of the joint may be generated (e.g., by surgical assistance system 100) based on medical images of the bones of the joint. Furthermore, in this example, surgical assistance system 100 may virtually move the 3D virtual models of the bones in accordance with the position data so that the 3D virtual models of the bones have the same spatial relationship as the real bones of the joint at the plurality of positions.

Surgical assistance system 100 (e.g., joint tension unit 410) may generate joint tension data 418 based on the positions of the bones of the joint (704). For instance. surgical assistance system 100 may determine distances between the bones based on data from sensors of MR visualization device 104 as discussed elsewhere in this disclosure. Furthermore, surgical assistance system 100 (e.g., plan modification unit 412) may determine, based on the joint tension data 418, areas of a target bone to remove (706). The target bone is one of the bones of the joint. For instance, surgical assistance system 100 may determine the areas of the target bone to remove based on a predetermined mapping of distances between the bones to areas of the bone to remove.

In the example of FIG. 7, surgical assistance system 100 (e.g., registration unit 408) may generate registration data (e.g., registration data 416) that registers markers 118 with a coordinate system (708). The markers are attached to one or more of the bones of the joint. For instance, surgical assistance system 100 may generate the registration data in accordance with any of the examples provided elsewhere in this disclosure.

Based on the registration data, surgical assistance system 100 (e.g., robot control unit 414) may control operation of robotic arm 110, e.g., during removal of bone tissue from the areas of the target bone (710). For example. a virtual surgical plan may indicate coordinates in a first coordinate system of locations on a virtual model of the target bone to remove. In this example, surgical assistance system 100 may use the registration data to translate the coordinates in the first coordinate system to coordinates in a coordinate system representing real world objects, such as surgical tool 112 and the target bone. Based on the coordinates in the coordinate system representing real world objects, surgical assistance system 100 may control robotic arm 110, e.g., to move robotic arm 110 in accordance with the surgical plan. to respond to certain movements by user 108 of surgical tool 112, and so on.

The following is a non-limiting list of aspects that are in accordance with one or more techniques of this disclosure.

Aspect 1: A computer-implemented method for assisting an orthopedic surgery includes obtaining, by a surgical assistance system. position data generated based on signals from one or more sensors of a mixed-reality (MR) visualization device while bones of a joint are at a plurality of positions, wherein the MR visualization device is worn by a user; determining, by the surgical assistance system, based on the position data, positions of the bones of the joint; generating, by the surgical assistance system, joint tension data based on the positions of the bones of the joint; determining, by the surgical assistance system, based on the joint tension data, areas of a target bone to remove, wherein the target bone is one of the bones of the joint; generating, by the surgical assistance system, registration data that registers markers with a coordinate system, wherein the markers are attached to one or more of the bones of the joint; and based on the registration data, controlling, by the surgical assistance system, operation of a robotic arm of a robot during removal of bone tissue from the areas of the target bone.

Aspect 2: The method of aspect 1, wherein the MR visualization device is a first MR visualization device and the method further comprises causing at least one of the first MR visualization device or a second MR visualization device to present a virtual guide overlaid on the target bone, the virtual guide indicating the areas of bone to remove.

Aspect 3: The method of any of aspects 1 and 2, wherein generating the registration data comprises generating the registration data based on signals from sensors of the robotic arm.

Aspect 4: The method of any of aspects 1 through 3, wherein determining the positions of the bones comprises determining, based on the position data, positions of 3D virtual models of the bones.

Aspect 5: The method of any of aspects 1 through 4, wherein generating the joint tension data comprises determining distances between the bones for each of the positions.

Aspect 6: The method of any of aspects 1 through 5, wherein the positions are along a direction of motion of the joint.

Aspect 7: The method of any of aspects 1 through 6, wherein controlling operation of the robotic arm comprises causing the robotic arm to respond to an attempt by the user to remove bone tissue in other areas of the bone.

Aspect 8: A surgical assistance system includes a memory configured to store registration data; and processing circuitry configured to: obtain position data generated based on signals from one or more sensors of a mixed-reality (MR) visualization device while bones of a joint are at a plurality of positions, wherein the MR visualization device is worn by a user; determine, based on the position data, positions of the bones of the joint; generate joint tension data based on the positions of the bones of the joint; determine, based on the joint tension data, areas of a target bone to remove, wherein the target bone is one of the bones of the joint; generate the registration data, wherein the registration data registers markers with a coordinate, wherein the markers are attached to one or more of the bones of the joint; and based on the registration data, control operation of a robotic arm of a robot during removal of bone tissue from the areas of the target bone.

Aspect 9: The surgical assistance system of aspect 8, wherein the MR visualization device is a first MR visualization device and the processing circuitry is further configured to cause at least one of the first MR visualization device or a second MR visualization device to present a virtual guide overlaid on the target bone, the virtual guide indicating the areas of bone to remove.

Aspect 10: The surgical assistance system of any of aspects 8 and 9, wherein the processing circuitry is configured to, as part of generating the registration data, generate the registration data based on signals from sensors of the robotic arm.

Aspect 11: The surgical assistance system of any of aspects 8 through 10, wherein the processing circuitry is configured to, as part of determining the positions of the bones, determine, based on the position data, positions of 3D virtual models of the bones.

Aspect 12: The surgical assistance system of any of aspects 8 through 11, wherein the processing circuitry is configured to, as part of generating the joint tension data, determine distances between the bones for each of the positions.

Aspect 13: The surgical assistance system of any of aspects 8 through 12, wherein the positions are along a direction of motion of the joint.

Aspect 14: The surgical assistance system of any of aspects 8 through 13, wherein the processing circuitry is configured to, as part of controlling operation of the robotic arm, cause the robotic arm to respond to an attempt by the user to remove bone tissue in other areas of the bone.

Aspect 15: The surgical assistance system of any of aspects 8 through 14, further comprising at least one of the robotic arm and the MR visualization device.

Aspect 16: A computing system comprising means for performing the methods of any of aspects 1-7.

Aspect 17: A computer-readable data storage medium having instructions stored thereon that, when executed, cause a computing system to perform the methods of any of aspects 1-7.

While the techniques been disclosed with respect to a limited number of examples, those skilled in the art, having the benefit of this disclosure, will appreciate numerous modifications and variations there from. For instance, it is contemplated that any reasonable combination of the described examples may be performed. It is intended that the appended claims cover such modifications and variations as fall within the true spirit and scope of the invention.

It is to be recognized that depending on the example, certain acts or events of any of the techniques described herein can be performed in a different sequence, may be added, merged, or left out altogether (e.g., not all described acts or events are necessary for the practice of the techniques). Moreover, in certain examples, acts or events may be performed concurrently, e.g., through multi-threaded processing, interrupt processing, or multiple processors, rather than sequentially.

In one or more examples, the functions described may be implemented in hardware, software, firmware, or any combination thereof. If implemented in software, the functions may be stored on or transmitted over as one or more instructions or code on a computer-readable medium and executed by a hardware-based processing unit. Computer-readable media may include computer-readable storage media, which corresponds to a tangible medium such as data storage media, or communication media including any medium that facilitates transfer of a computer program from one place to another. e.g., according to a communication protocol. In this manner, computer-readable media generally may correspond to (1) tangible computer-readable storage media which is non-transitory or (2) a communication medium such as a signal or carrier wave. Data storage media may be any available media that can be accessed by one or more computers or one or more processors to retrieve instructions, code and/or data structures for implementation of the techniques described in this disclosure. A computer program product may include a computer-readable medium.

By way of example, and not limitation, such computer-readable storage media can comprise RAM, ROM, EEPROM, CD-ROM or other optical disk storage, magnetic disk storage, or other magnetic storage devices, flash memory, or any other medium that can be used to store desired program code in the form of instructions or data structures and that can be accessed by a computer. Also, any connection is properly termed a computer-readable medium. For example, if instructions are transmitted from a website, server, or other remote source using a coaxial cable, fiber optic cable. twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. It should be understood, however, that computer-readable storage media and data storage media do not include connections, carrier waves, signals, or other transitory media, but are instead directed to non-transitory, tangible storage media. Disk and disc, as used herein, includes compact disc (CD), laser disc, optical disc, digital versatile disc (DVD), floppy disk and Blu-ray disc, where disks usually reproduce data magnetically, while discs reproduce data optically with lasers. Combinations of the above should also be included within the scope of computer-readable media.

Operations described in this disclosure may be performed by one or more processors, which may be implemented as fixed-function processing circuits, programmable circuits, or combinations thereof, such as one or more digital signal processors (DSPs), general purpose microprocessors, application specific integrated circuits (ASICs), field programmable gate arrays (FPGAs), or other equivalent integrated or discrete logic circuitry. Fixed-function circuits refer to circuits that provide particular functionality and are preset on the operations that can be performed. Programmable circuits refer to circuits that can programmed to perform various tasks and provide flexible functionality in the operations that can be performed. For instance, programmable circuits may execute instructions specified by software or firmware that cause the programmable circuits to operate in the manner defined by instructions of the software or firmware. Fixed-function circuits may execute software instructions (e.g., to receive parameters or output parameters), but the types of operations that the fixed-function circuits perform are generally immutable. Accordingly, the terms “processor” and “processing circuitry,” as used herein may refer to any of the foregoing structures or any other structure suitable for implementation of the techniques described herein.

Various examples have been described. These and other examples are within the scope of the following claims.

1. A computer-implemented method for assisting an orthopedic surgery, the method comprising:
obtaining, by a surgical assistance system, position data generated based on signals from one or more sensors of a mixed-reality (MR) visualization device while bones of a joint are at a plurality of positions, wherein the MR visualization device is worn by a user;
determining, by the surgical assistance system, based on the position data, positions of the bones of the joint;
generating, by the surgical assistance system, joint tension data based on the positions of the bones of the joint;
determining, by the surgical assistance system, based on the joint tension data, areas of a target bone to remove, wherein the target bone is one of the bones of the joint;
generating, by the surgical assistance system, registration data that registers markers with a coordinate system, wherein the markers are attached to one or more of the bones of the joint; and
based on the registration data, controlling, by the surgical assistance system, operation of a robotic arm of a robot during removal of bone tissue from the areas of the target bone.

2. The method of claim 1, wherein the MR visualization device is a first MR visualization device and the method further comprises causing at least one of the first MR visualization device or a second MR visualization device to present a virtual guide overlaid on the target bone, the virtual guide indicating the areas of the target bone to remove.

3. The method of claim 1, wherein generating the registration data comprises generating the registration data based on signals from sensors of the robotic arm.

4. The method of claim 1, wherein determining the positions of the bones comprises determining, based on the position data, positions of 3D virtual models of the bones.

5. The method of claim 1, wherein generating the joint tension data comprises determining distances between the bones for each of the positions in the plurality of positions.

6. The method of claim 1, wherein the positions in the plurality of positions are along a direction of motion of the joint.

7. The method of claim 1, wherein controlling operation of the robotic arm comprises causing the robotic arm to respond to an attempt by the user to remove bone tissue in other areas of the target bone.

8. A surgical assistance system comprising:
a memory configured to store registration data; and
processing circuitry configured to:
obtain position data generated based on signals from one or more sensors of a mixed-reality (MR) visualization device while bones of a joint are at a plurality of positions, wherein the MR visualization device is worn by a user;
determine, based on the position data, positions of the bones of the joint;
generate joint tension data based on the positions of the bones of the joint;
determine, based on the joint tension data, areas of a target bone to remove, wherein the target bone is one of the bones of the joint;
generate the registration data, wherein the registration data registers markers with a coordinate, wherein the markers are attached to one or more of the bones of the joint; and
based on the registration data, control operation of a robotic arm of a robot during removal of bone tissue from the areas of the target bone.

9. The surgical assistance system of claim 8, wherein the MR visualization device is a first MR visualization device and the processing circuitry is further configured to cause at least one of the first MR visualization device or a second MR visualization device to present a virtual guide overlaid on the target bone, the virtual guide indicating the areas of the target bone to remove.

10. The surgical assistance system of claim 8, wherein the processing circuitry is configured to, as part of generating the registration data, generate the registration data based on signals from sensors of the robotic arm.

11. The surgical assistance system of claim 8, wherein the processing circuitry is configured to, as part of determining the positions of the bones, determine, based on the position data, positions of 3D virtual models of the bones.

12. The surgical assistance system of claim 8, wherein the processing circuitry is configured to, as part of generating the joint tension data, determine distances between the bones for each of the positions in the plurality of positions.

13. The surgical assistance system of claim 8, wherein the positions in the plurality of positions are along a direction of motion of the joint.

14. The surgical assistance system of claim 8, wherein the processing circuitry is configured to, as part of controlling operation of the robotic arm, cause the robotic arm to respond to an attempt by the user to remove bone tissue in other areas of the target bone.

15. The surgical assistance system of claim 8, further comprising at least one of the robotic arm and the MR visualization device.

16. (canceled)

17. A non-transitory computer-readable data storage medium having instructions stored thereon that, when executed, cause a computing system to;
obtain position data generated based on signals from one or more sensors of a mixed-reality (MR) visualization device while bones of a joint are at a plurality of positions, wherein the MR visualization device is worn by a user;
determine, based on the position data, positions of the bones of the joint;
generate joint tension data based on the positions of the bones of the joint;
determine, based on the joint tension data, areas of a target bone to remove, wherein the target bone is one of the bones of the joint;
generate registration data that registers markers with a coordinate system, wherein the markers are attached to one or more of the bones of the joint; and
based on the registration data, control operation of a robotic arm of a robot during removal of bone tissue from the areas of the target bone.
